(19) United States
(12) Patent Application Publication
(10) Pub. No.: US 2026/0256484 A1
(43) Pub. Date: Sep. 3, 2026

(54) THROMBECTOMY APPARATUS AND METHOD UTILIZING MAGNETICALLY RESPONSIVE EXPANDABLE SEGMENTS

(71) Applicant: Arizona Board of Regents on behalf of Arizona State University, Scottsdale, AZ (US)

(72) Inventors: Hamidreza Marvi, Chandler, AZ (US); Tao Zhang, Tempe, AZ (US); Morteza Varzaghan, Tempe, AZ (US); Deepit Arora, Tempe, AZ (US)

(21) Appl. No.: 19/552,486

(22) Filed: Feb. 27, 2026

Related U.S. Application Data
(60) Provisional application No. 63/766,185, filed on Mar. 3, 2025.

(51) Int. Cl. A61B 17/22; A61B 17/00

(52) U.S. Cl. CPC A61B 17/22031; A61B 17/00234; A61B 2017/00367; A61B 2017/00778; A61B 2017/00876; A61B 2017/22034

(57) ABSTRACT

A thrombectomy apparatus and associated method that permit removal of a blood clot from a blood vessel utilize multiple segments (i.e., a first terminal segment and additional segment(s)), with each segment including magnetically responsive element(s) configured to cooperate with the at least one magnetic field supplied by magnetic field generator(s) arranged external to an animal body. Each segment includes projections expandable end portion(s) thereof, such that when adjacent segments are magnetically manipulated to press against one another (e.g., forming a mated segment assembly), the projections deflect outwardly and thereby increase a maximum width of the end portions. A method includes moving a terminal segment and at least one additional segment in one direction through a blood clot, pressing the segments against one another to expand terminal end segments and form a mated segment assembly, and moving the assembly in an opposing direction to engage and dislodge the clot.

### CROSS-REFERENCE TO RELATED APPLICATION(S)

This application claims priority to U.S. Provisional Patent Application No. 63/766,185 filed on March 3, 2025, wherein the entire contents of the foregoing application are hereby incorporated by reference herein.

### TECHNICAL FIELD

This disclosure relates to a magnetically operable thrombectomy apparatus and method to dislodge blood clots from blood vessels for therapeutic treatment.

### BACKGROUND

Stroke and other thrombotic conditions represent a significant global health burden. Current thrombotic treatments often involve invasive medical interventions such as surgical or mechanical thrombectomies, which are physically demanding on a patient’s body. Alternatively, pharmacological approaches may be utilized without surgical intervention, but such approaches carry risks of incomplete clot removal or other systemic side effects. Existing thrombectomy methods are limited in their ability to navigate complex vascular networks and in their ability to adapt dynamically to clot morphology.

In view of the foregoing, the art continues to seek improvement in thrombectomy apparatuses and methods to overcome the limitations of existing treatments.

### SUMMARY

Aspects of the present disclosure relate to a thrombectomy apparatus deployable within an animal body and an associated method for manipulating (e.g., moving or positioning) the thrombectomy apparatus during a surgical procedure, with magnetically responsive segments of the thrombectomy apparatus configured to be delivered to a target vascular region using a catheter or similar surgical device. In certain implementations, at least one magnetic field source (e.g., a permanent magnet, a ferroelectric magnet, or an electromagnet) arranged external to the animal body (encompassing a body of a human or non-human animal) is moved using at least one robotic actuator, and at least one magnetic field generated by the at least one magnetic field source is used to alter position of one or more segments of the plurality of segments. An exemplary thrombectomy apparatus may include a plurality of segments, including a first terminal segment and one or more additional segments, wherein each segment includes one or more magnetically responsive elements configured to cooperate with the at least one magnetic field supplied by magnetic field generator(s) arranged external to the animal body to permit manipulation of the thrombectomy apparatus. Each segment includes a plurality of projections at one or two expandable end portions thereof, such that when adjacent segments are magnetically manipulated within a blood vessel and press against one another (e.g., to form a mated segment assembly), the projections of end portions of adjacent segments are deflected outwardly relative to longitudinal axes of the segments, thereby increasing a maximum width dimension (e.g., diameter) of the expandable end portions of each segment relative to a non-expanded width dimension thereof. The expanded width provided by outwardly deflected projections of adjacent segments permits the mated segment assembly to engage a blood clot so that the engaged blood clot can be dislodged (and removed) from a blood vessel by magnetic manipulation of the mated segment assembly.

In one aspect, the disclosure relates to a thrombectomy apparatus configured to permit removal of a blood clot from a blood vessel, the apparatus comprising: a plurality of segments configured to be moved within a blood vessel of an animal body responsive to application of magnetic fields by one or more magnetic field generators external to the animal body, wherein the plurality of segments comprises: a first terminal segment comprising at least one first magnetically responsive element arranged within a first terminal segment body structure having a first terminal segment longitudinal axis, the first terminal segment body structure comprising a tapered end portion and an expandable terminal segment end portion that opposes the tapered end portion, the expandable terminal segment end portion comprising a plurality of projections extending substantially parallel to the first terminal segment longitudinal axis; and at least one additional segment, wherein each additional segment of the at least one additional segment comprises at least one second magnetically responsive element arranged within an additional segment body structure having an additional segment longitudinal axis and at least one expandable additional segment end portion that comprises a plurality of projections extending substantially parallel to the additional segment longitudinal axis; wherein upon magnetic manipulation of the first terminal segment and an additional segment of the at least one additional segment to press against one another, the plurality of projections of the expandable terminal segment end portion are configured to cooperate with the plurality of projections of the at least one expandable additional segment end portion to deflect outwardly relative to the first terminal segment longitudinal axis and the additional segment longitudinal axis, respectively.

In certain embodiments, the at least one magnetically responsive element of the first terminal segment and/or the at least one second magnetically responsive element of the at least one additional segment is spherical or cylindrical in shape.

In certain embodiments, the at least one additional segment comprises multiple additional segments, and upon magnetic manipulation of each additional segment of the multiple additional segments to press against one another along expandable segment end portions thereof, the plurality of projections of each additional segment are configured to cooperate with the plurality of projections of an adjacent additional segment to deflect outwardly relative to the respective additional segment longitudinal axis.

In certain embodiments, the at least one additional segment comprises a second terminal segment for which the additional segment body structure comprises a second terminal segment body structure having the additional segment longitudinal axis, the second terminal segment body structure comprising a second tapered end portion that opposes the at least one expandable additional segment end portion; and upon magnetic manipulation of the first terminal segment and the second terminal segment to contact and press against one another, the plurality of projections of the expandable terminal segment end portion of the first terminal segment are configured to cooperate with the plurality of projections of the at least one expandable additional segment end portion of the second terminal segment to deflect outwardly relative to the first terminal segment longitudinal axis and the additional segment longitudinal axis, respectively. In certain embodiments, magnetic interactions between magnetic elements within adjacent segments provide sufficient force to maintain contact between the adjacent segments and to cause deflection of projections of the segments at an interface therebetween.

In certain embodiments, the at least one additional segment comprises at least two additional segments, the at least two additional segments including a second terminal segment and at least one intermediate segment that is arranged to be positioned between the first terminal segment and the second terminal segment; the additional segment body structure of the second terminal segment comprises a second terminal segment body structure having the additional segment longitudinal axis, the second terminal segment body structure comprising a second tapered end portion that opposes the at least one additional segment end portion thereof; and upon magnetic manipulation of the first terminal segment, the at least one intermediate segment, and the second terminal segment to press against one another, the plurality of projections of the expandable terminal segment end portion of the first terminal segment are configured to cooperate with the plurality of projections of one expandable additional segment end portion of the at least one intermediate segment to deflect outwardly relative to the first terminal segment longitudinal axis, and the plurality of projections of one expandable additional segment end portion of the second terminal segment are configured to cooperate with the plurality of projections of another expandable segment end portion of the at least one intermediate segment to deflect outwardly relative to the additional segment longitudinal axis.

In certain embodiments, the tapered end portion of the first terminal segment is formed from a material having a durometer value greater than a durometer value of a material forming the first terminal segment body structure of the first terminal end segment.

In certain embodiments, the tapered end portion of the second terminal segment is formed from a material having a durometer value greater than a durometer value of a material forming the second terminal segment body structure of the second terminal end segment.

In another aspect, the disclosure relates to a method of removing a blood clot from a blood vessel of an animal body utilizing a thrombectomy apparatus according to claim 1, the method comprising: identifying a target vasculature region of the animal body including a blood vessel containing the blood clot; inserting the first terminal segment into the target vasculature region; moving, by the one or more magnetic field generators external to the animal body, the first terminal segment within the blood vessel and through the blood clot in a first direction; inserting the at least one additional segment into the target vasculature region; moving, by the one or more magnetic field generators, the at least one additional segment within the blood vessel and through the blood clot in the first direction to a position proximate to the first terminal segment and causing the first terminal segment and the at least one additional segment to press against one another, thereby causing the pluralities of projections thereof to deflect outwardly relative to the first terminal segment longitudinal axis and the additional segment longitudinal axis to expand the expandable terminal end segment portion and to expand the at least one expandable additional segment end portion, respectively, and to yield a mated segment assembly; and moving, by the one or more magnetic field generators, the mated segment assembly in a second direction that opposes the first direction, to cause the outwardly deflected pluralities of projections to engage the blood clot and dislodge at least a portion of the blood clot from the blood vessel.

In certain embodiments, the inserting of the first terminal segment into the vasculature region further comprises: orienting, prior to insertion, the first terminal segment such that the tapered end portion faces the blood clot in the first direction.

In certain embodiments, the at least one additional segment comprises at least two additional segments, the at least two additional segments comprising a second terminal segment and at least one intermediate segment that is arranged to be positioned between the first terminal segment and the second terminal segment; the additional segment body structure of the second terminal segment comprises a second terminal segment body structure having the additional segment longitudinal axis, the second terminal segment body structure comprising a second tapered end portion that opposes the at least one expandable terminal segment end portion thereof; and the inserting of the at least one additional segment into the target vasculature region further comprises: inserting the at least one intermediate segment into the target vascular region prior to inserting the second terminal segment into the target vascular region.

In certain embodiments, the inserting of the second terminal segment further comprises: prior to inserting the second terminal segment into the target vascular region, orienting the second terminal segment such that the tapered end portion faces away from the blood clot.

In certain embodiments, each field generator of the one or more external field generators comprises a pair of magnetic field sources, the pair of magnetic field sources diametrically opposed to one another around the target vasculature region.

In another aspect, any one or more aspects or features described herein may be combined with any one or more other aspects or features for additional advantage.

Other aspects and embodiments will be apparent from the detailed description and accompanying drawings.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWINGS

FIG. 1 is a perspective view of a thrombectomy apparatus including multiple intermediate segments arranged between two terminal segments with projections of the segments in an outwardly deflected state to form a mated segment assembly according to a first embodiment of the invention.

FIG. 2 is a perspective view of a first terminal segment according to the first embodiment.

FIG. 3 is a perspective view of an intermediate segment according to the first embodiment.

FIG. 4 is a side cross-sectional view of the first terminal segment of FIG. 2.

FIG. 5 is a side cross-sectional view of an intermediate segment of FIG. 3.

FIG. 6A is perspective view of the first terminal segment and an intermediate segment aligned with, but not contacting, one another according to the first embodiment.

FIG. 6B is a partial perspective view of the first terminal segment and a portion of an intermediate segment contacting one another with projections of the segments in an outwardly deflected state according to the first embodiment.

FIG. 7 is a schematic view of a thrombectomy apparatus within a blood vessel at several stages of deployment, according to the first embodiment.

FIG. 8 is a perspective view of an intermediate segment with projections in an outwardly deflected state according to the first embodiment.

FIG. 9 is a flow diagram outlining steps of a method of using a thrombectomy apparatus as disclosed herein.

FIG. 10 is a flow diagram outlining steps of another method of using a thrombectomy apparatus as disclosed herein.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term "and/or" includes any and all combinations of one or more of the associated listed items.

It will be understood that when an element such as a layer, region, or substrate is referred to as being "on" or extending "onto" another element, it can be directly on or extend directly onto the other element or intervening elements may also be present. In contrast, when an element is referred to as being "directly on" or extending "directly onto" another element, there are no intervening elements present. Likewise, it will be understood that when an element such as a layer, region, or substrate is referred to as being "over" or extending "over" another element, it can be directly over or extend directly over the other element or intervening elements may also be present. In contrast, when an element is referred to as being "directly over" or extending "directly over" another element, there are no intervening elements present. It will also be understood that when an element is referred to as being "connected" or "coupled" to another element, it can be directly connected or coupled to the other element or intervening elements may be present. In contrast, when an element is referred to as being "directly connected" or "directly coupled" to another element, there are no intervening elements present.

Relative terms such as "below" or "above" or "upper" or "lower" or "horizontal" or "vertical" may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms "a," "an," and "the" are intended to include the plural forms as well, unless the context clearly indicates otherwise. It will be further understood that the terms "comprises," "comprising," "includes," and/or "including" when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Aspects of the present disclosure relate to a thrombectomy apparatus deployable within an animal body and an associated method for manipulating (e.g., moving or positioning) the thrombectomy apparatus during a surgical procedure, with magnetically responsive segments of the thrombectomy apparatus configured to be delivered to a target vascular region using a catheter or similar surgical device. In certain implementations, at least one magnetic field source (e.g., a permanent magnet, a ferroelectric magnet, or an electromagnet) arranged external to the animal body is moved using at least one robotic actuator, and at least one magnetic field generated by the at least one magnetic field source is used to alter position of one or more segments of the plurality of segments (whether individually or as a mated segment assembly). An exemplary thrombectomy apparatus may include a plurality of segments including a first terminal segment and one or more additional segments, wherein each segment includes one or more magnetically responsive elements configured to cooperate with the at least one magnetic field supplied by magnetic field generator(s) arranged external to the animal body to permit manipulation of the segments, whether individually or joined to form a mated segment assembly. The one or more additional segments may include a second terminal segment, or may include a second terminal segment and one or more intermediate segments. As used herein, the term “animal body” is intended to encompass a body of a human or non-human animal (e.g., a mammal, reptile, etc.).

In certain embodiments, a target vasculature region containing a blood clot may be identified by utilizing conventional imaging techniques, such as ultrasound imaging, computed tomography (CT) scans, magnetic resonance imaging (MRI), or the like. Segments of a magnetically responsive thrombectomy apparatus may be deployed into the target vasculature region in the vicinity of the blood clot (e.g., in a blood vessel containing the blood clot) and assembled into a mated segment assembly. The segments of the thrombectomy apparatus may be deployed (e.g., using a catheter or similar instrument) either upstream of the clot (relative to the flow of blood in the vasculature) or, more preferably, downstream of the clot. One or more magnetic field sources may be used to effectuate movement and manipulation of individual segments following their insertion into the blood vessel. The one or more magnetic field sources may include a permanent magnet, a ferroelectric magnet, an electromagnet, or the like. The one or more magnetic field sources may be mounted on a distal end of a robotic actuator. By using the robotic actuator, one or more segments may be moved in a blood vessel. In certain embodiments, individual segments are moved through the blood clot in a first direction, then joined together to form a mated segment assembly (with projections arranged at end segments of contacting segments being deflected outward into an expanded state to provide an increased maximum width dimension), and the mated segment assembly is moved in an opposing second direction to engage the blood clot so that the blood clot can be dislodged from the blood vessel and removed (e.g., using a catheter or similar instrument)..

A thrombectomy apparatus comprises a plurality of segments including a first terminal segment and at least one additional segment. In certain embodiments, the at least one additional segments may include at least two additional segments, at least three additional segments, at least four additional segments, or at least n additional segments. Each of the plurality of segments includes a body structure formed of a suitable biocompatible material (e.g., silicone, thermoplastic elastomer, etc.). A body structure of a segment may be sized and shaped to permit passage through a catheter or similar instrument, and to permit passage through a blood vessel. In certain embodiments, at least a portion of a body structure may be substantially cylindrical, conical, frustoconical, cuboid, or rectangular cuboid in shape. The body structure of each segment contains one or more magnetically responsive elements.

In certain embodiments, one or more magnetically responsive elements contained within a body structure of a segment comprise cylindrical permanent magnets, spherical permanent magnets, or ferromagnetic particles suspended in body structure material. The magnetically responsive elements are configured to respond to external magnetic fields generated by one or more magnetic field sources. In certain embodiments, one or more magnetically responsive elements may be overmolded, embedded within, adhered to, or otherwise fixed to a body structure of a segment as disclosed herein.

A first terminal segment may include a tapered end portion on one end of a first terminal segment body structure and an expandable terminal segment end portion at an opposite end. The tapered end portion tapers to a width or diameter that is smaller than a corresponding width or diameter of the first terminal segment body structure, optionally tapering continuously toward an end point. In certain embodiments, the tapered end portion may be used to pierce a blood clot to form an opening therein to permit passage of the first terminal segment (with the same opening being useable to permit subsequent passage of additional segments). The tapered end portion may be formed of the same material or, more preferably, a different material than the first terminal segment body structure such as a thermoplastic elastomer (TPE). Consistent with the desired functionality of piercing a blood clot, in certain embodiments the tapered portion maybe formed of a material that is less elastic (e.g., more rigid) than a non-tapered portion of the terminal segment, such as of a material having higher durometer value (e.g., on a Shore A or Shore D scale) the material of remainder of the first terminal segment body structure. In certain embodiments, the tapered portion may be adhered, overmolded, or otherwise fixed to a remainder of first terminal segment body structure. The expandable terminal segment end portion (which opposes the tapered end portion) includes a plurality of projections integrally formed thereon and extending substantially parallel to a longitudinally axis of the first terminal segment. The plurality of projections may extend in a longitudinal direction and be radially distributed proximate to an outer border of the first terminal segment body structure. Spaces or voids are provided between at least portions of adjacent projections. When the terminal segment is magnetically manipulated and brought into contact with an additional segment (e.g., an intermediate segment or another terminal segment, also having a plurality of projections) within a blood vessel, the plurality of projections of the respective adjacent segments are configured to cooperate with one another and be deflected outward relative to longitudinal axes of the respective segments. In certain embodiments, the deflected pluralities of projections may be arranged substantially orthogonal to longitudinal axes of the respective adjacent segments.

As noted previously herein, a first terminal segment is configured to cooperate with one or more additional segments to form a mated segment assembly within a blood vessel. In certain embodiments, the one or more additional segments may include a second terminal segment, or may include a second terminal segment in combination with at least one intermediate segment (with the intermediate segment(s) being positionable between terminal end segments). Each additional segment (whether a second terminal segment or an intermediate segment) includes a body structure (i.e., “additional segment body structure”) and includes at least one expandable additional segment end portion having a plurality of projections integrally formed thereon and extending substantially parallel to an additional segment longitudinal axis of the additional segment body structure. In embodiments wherein the additional segment(s) include one or more intermediate segments, each intermediate segment includes two (opposing) expandable additional segment end portions each having a plurality of projections. Each plurality of projections is configured to cooperate with a plurality of projections of an adjacent segment (e.g., another intermediate segment or a terminal segment) to deflect outwardly relative to longitudinal axes of the adjacent segments when the adjacent segments are pressed against one another (e.g., by application of at least one magnetic field).

In certain embodiments, the at least one additional segment includes a second terminal segment, wherein a second terminal segment may be substantially identical to a first terminal segment as described hereinabove, but may be inserted into a blood vessel with an orientation opposing that of a first terminal segment. The description herein of a first terminal segment is equally applicable to a second terminal segment and is hereby incorporated by reference.

In certain embodiments, the first terminal segment and the at least one additional segment are configured to be manipulated by magnetic fields (generated by one or more magnetic field sources external to an animal body) into proximity with one another within a blood vessel and to press against one another (e.g., by interaction of magnetic elements within adjacent segments). In certain embodiments, one magnetic field source may be used to move, or maintain position of, one (e.g., first terminal) segment, and another magnetic field source may be used to move at least one additional (e.g., intermediate or second terminal) to cause the respective segments to contact (and thereby press against) one another within a blood vessel. Contact pressure between adjacent segments may be provided and maintained by interaction between internal magnetic elements of adjacent segments. As a result of being pressed together, the plurality of projections of the first terminal segment cooperate with the plurality of projections of the at least one additional segment to deflect outwardly relating to longitudinal axes of the adjacent segments. In certain embodiments, the first terminal segment and the at least one additional segment are configured to be magnetically coupled (and pressed against) to one another by interaction of their internal magnetic elements when the respective segments are arranged in contact with one another.

In certain embodiments, the first terminal segment is configured to be oriented such that the tapered end portion faces towards the blood clot during insertion into a blood vessel and the expandable terminal segment end portion faces away from the blood clot during insertion. This orientation permits the tapered end portion to pierce a blood clot when the first terminal segment is magnetically moved in a first direction. For embodiments in which the at least one additional segment includes a second terminal segment, the second terminal segment may be oriented such that a tapered end portion thereof faces away from the blood clot (and the expandable terminal segment end portion faces toward the blood clot) during insertion. This orientation of the second terminal segment may assist in positioning a mated segment assembly (including the second terminal segment) within an opening pre-defined in a blood clot when the mated segment assembly is moved in the second direction to positively engage the blood clot to dislodge it from the blood vessel.

In certain embodiments, a magnetic field source includes a pair of complementary magnetic field source generators located at diametrically opposing positions relative to a blood vessel of the target vasculature region. This arrangement permits balanced magnetic forces to be applied to one or more segments (or a mated segment assembly) in a desired direction, without application of net force in a direction that would tend to pull a segment directly toward a single magnetic field source generator. In certain embodiments, multiple pairs of complementary magnetic field source generators may be provided. In certain embodiments, each pair of diametrically opposed magnetic field source generators may be supported by a dedicated robotic actuator.

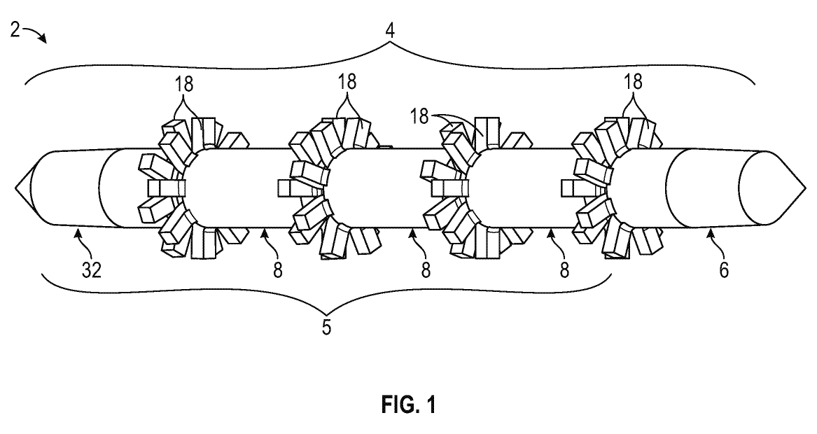

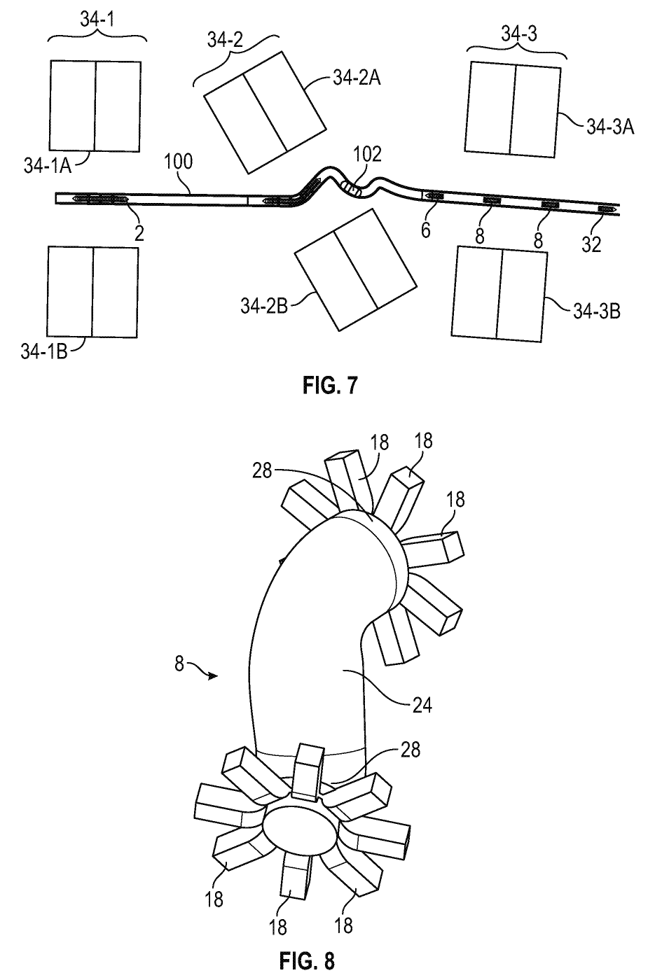

With references to the figures, FIG. 1 shows a thrombectomy apparatus comprising a plurality of segments 4 that are pressed against one another to form a mated segment assembly 2. The plurality of segments 4 includes a first terminal segment 6 and multiple additional segments 5, which include a second terminal segment 32 and three intermediate segments 8. At interfaces where the various segments 6, 8, 32 contact one another, each segment 6, 8, 32 includes multiple projections 18 that cooperate with projections 18 of an adjacent segment. As shown in FIG. 1, the projections 18 are deflected outwardly (e.g., relative to a central longitudinal axis of the mated segment assembly 2) to provide localized regions of increased width (e.g., diameter) relative to other portions of the mated segment assembly 2. The segments 4 are configured to be moved (whether individually prior to formation of the mated segment assembly 2, or as a group following formation of the mated segment assembly 2) within a blood vessel 100 (e.g., 100 in FIG. 7) responsive to application of one or more magnetic fields that may be applied to one or more magnetic field generators (e.g., 34-1 to 34-3 in FIG. 7) arranged external to an animal body containing the blood vessel.

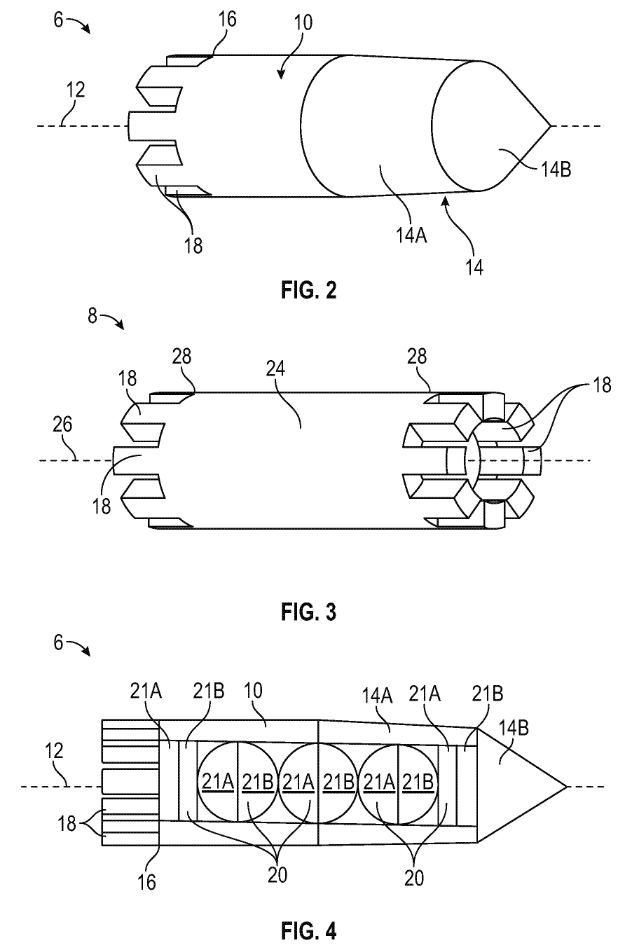

FIG. 2 is a perspective view, and FIG. 4 is a cross-sectional view, of the first terminal segment 6 shown in FIG. 1. As shown in FIG. 2 and FIG. 4, the first terminal segment 6 comprises a first terminal segment body structure 10 formed of a biocompatible and flexible material and having a generally cylindrical shape defining a first terminal segment longitudinal axis 12. The first terminal segment body structure 10 is configured to elastically deform and bend (to facilitate navigation of the first terminal segment 6 within a blood vessel (e.g., 100 in FIG. 7). The first terminal segment body structure 10 includes a tapered portion 14 at a first end portion of the first terminal segment body structure 10, and includes an expandable terminal segment end portion 16 at an opposing second end portion of the first terminal body structure 10.

The tapered end portion 14 includes at least one tapered region such that a surface of the tapered end portion 14 is angled towards the first terminal segment longitudinal axis 12 at the first end of the first terminal segment body structure 10. The tapered end portion 14 may include a first tapered region 14A and a second tapered region 14B (optionally coming to an end point), wherein surfaces of the first and the second tapered regions 14A, 14B are inclined at different angles relative to the first terminal segment longitudinal axis 12. In certain embodiments, the tapered end portion 14 may be formed of a material that is more rigid (less elastic) than the first terminal segment body structure 10, consistent with a desired function of the tapered end portion 14 to pierce a blood clot to form an opening therein.

The first terminal segment 10 additionally includes a plurality of projections 18 at the expandable terminal segment end portion 16. The plurality of projections 18 are preferably integral with the first terminal segment body structure 10, may be substantially parallel with the first terminal segment longitudinal axis 12 (when not in an expanded state), and may be radially distributed proximate to an outer border of the first terminal segment body structure 10.

As shown in FIG. 4, the first terminal segment 6 includes one or more first magnetically responsive elements 20 located within the first terminal segment body structure 10. The first magnetically responsive element(s) 20 may be disposed (e.g., linearly arranged) along the first terminal segment longitudinal axis 12 and each may be configured as a cylindrical permanent magnet and/or a spherical permanent magnet. Each first magnetically responsive element 20 may include a first polarity region 21A and a second polarity region 21B, wherein such polarity regions 21A, 21B may be provided in an alternating arrangement. Providing multiple first magnetically responsive elements 20 instead of a single (e.g., larger) first magnetically responsive element may enhance bendability of the first terminal segment 6 to permit the first terminal body structure 10 to flex or bend, which may beneficially enhance the ability of the first terminal segment (whether alone or as part of a mated segment assembly 2 in FIG. 1) to navigate a curved path through a blood vessel. In certain embodiments, the first magnetically responsive element(s) 20 may also be formed as ferromagnetic particles suspended in the first terminal segment body structure 10.

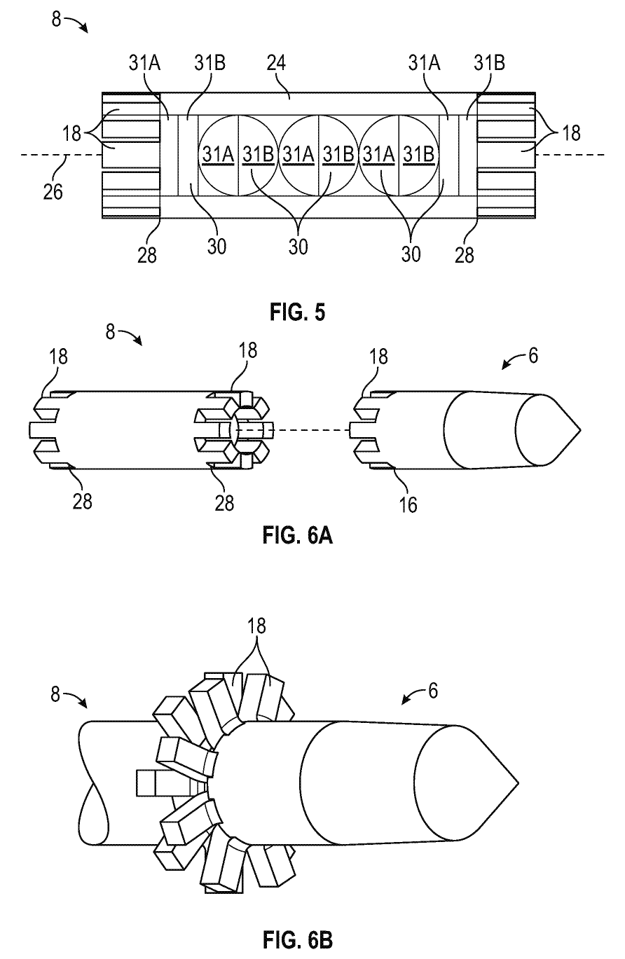

FIG. 3 is a perspective view, and FIG. 5 is a cross-sectional view, of an intermediate segment 8 as shown in FIG. 1. As shown in FIG. 3 and FIG. 5, the intermediate segment 8 includes an additional segment body structure 24 formed of a biocompatible and flexible material (e.g., silicone or another elastomeric material). The additional segment body structure 24 may have generally cylindrical shape defining an additional segment longitudinal axis 26. The additional segment body structure 24 is configured to elastically deform and bend (as shown in FIG. 8) to facilitate navigation of the at least one additional segment 8 within a blood vessel (e.g., 100 in FIG. 7). Additional segment end portions 28 are located at each opposing end of the additional segment body structure 24. Each additional segment end portion 28 includes a plurality of projections 18 that are preferably integral with the additional segment body structure 24 and (when not in an expanded state) are substantially parallel with the additional segment longitudinal axis 26. Each plurality of projections 18 is distributed proximate to an outer border of the additional segment body structure 24.

As shown in FIG. 5, the intermediate segment 8 includes one or more second magnetically responsive elements 30 within the additional segment body structure 24. The second magnetically responsive element(s) 30 may be disposed along the additional segment longitudinal axis 26 and each may be configured as a cylindrical permanent magnet and/or a spherical permanent magnet. Each second magnetically responsive element 30 may include a first polarity region 31A and a second polarity region 31B, wherein such polarity regions 31A, 31B may be provided in an alternating arrangement. Providing multiple second magnetically responsive elements 20 instead of a single (e.g., larger) second magnetically responsive element may enhance bendability of the additional segment 6 to permit the additional segment body structure 23 to flex or bend (e.g., as shown in FIG. 8), which may beneficially enhance the ability of the additional segment 6 (whether alone or as part of a mated segment assembly 2 in FIG. 1) to navigate a curved path through a blood vessel (e.g., 100 in FIG. 7). In certain embodiments, the at least one second magnetically responsive element 30 may also be formed as ferromagnetic particles suspended in the additional segment body structure 24.

In certain embodiments, at least one additional segment as recited in the appended claims may embody or include a second terminal segment 32 (shown in FIG. 1) that is substantially identical to the first terminal segment 6 illustrated and described in FIGS. 2 and 4.

As noted previously, multiple segments as described herein may be inserted into a blood vessel, conveyed in a first direction through a blood clot, and assembled to form a mated segment assembly (e.g., 2 in FIG. 1) that may be moved in a second direction to engage and dislodge the blood clot. Such segments may include a first terminal segment 6 according to FIG. 2 and at least one additional segment 5 as shown in FIG. 1 (e.g., at least one intermediate segment 8 according to FIG. 3, and/or a second terminal segment 32 as shown in FIG. 1). Upon magnetic manipulation of the first terminal segment 6 and an additional segment 5, the first terminal segment 6 and the additional segment 5 are configured to be magnetically coupled and pressed against another. When the at least one additional segment 5 comprises at least one intermediate segment 8 (shown in FIG. 3), the expandable terminal segment end portion 16 of the first terminal segment body structure 10 (shown in FIG. 2) is configured to press against the expandable additional segment end portion 28 of the at least one intermediate segment 8 (shown in FIG. 3). When the at least one additional segment 5 comprises a second terminal segment 32, the expandable terminal segment end portions 16 of respective first and second terminal segments 6, 32 are configured to be pressed together. When the at least one additional segment 8 comprises a second terminal segment 32 in addition to at least one intermediate segment 8, the expandable terminal segment end portion 16 of the first terminal segment body structure 10 (shown in FIG. 2) is configured to press against the expandable additional segment end portion 28 of at least one intermediate segment 8 (shown in FIG. 3), and the expandable terminal segment end portion 16 of the second terminal segment 32 (shown in FIG. 1) is configured to press against another expandable additional segment end portion 28 of an intermediate segment 8 (shown in FIG. 3) of the at least one intermediate segment 8.

FIG. 6A is perspective view of a first terminal segment 6 (according to FIG. 2) and an intermediate segment 8 (according to FIG. 3) aligned with, but not contacting, one another. FIG. 6A shows multiple projections 18 of each segment 6, 8 being in a relaxed, first state in which the projections are generally parallel to longitudinal axes of the respective segments 6, 8. FIG. 6B shows the first terminal segment 6 and a portion of the intermediate segment 8 contacting one another, with projections 18 of the segments 6, 8 in an outwardly deflected, second state in which the projections 18 are non-parallel to longitudinal axes of the respective segments 6, 8. In certain embodiments, the projections 18 when deflected may be substantially orthogonal to longitudinal axes of the respective segments 6, 8.

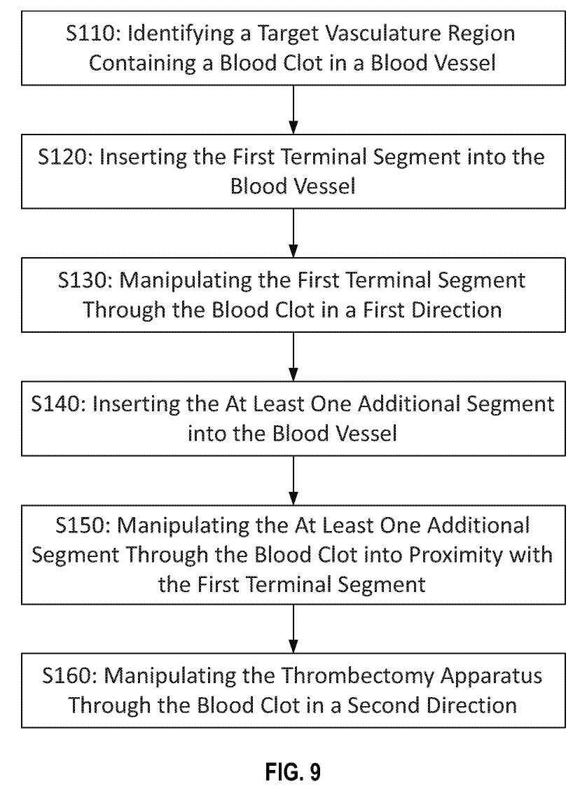

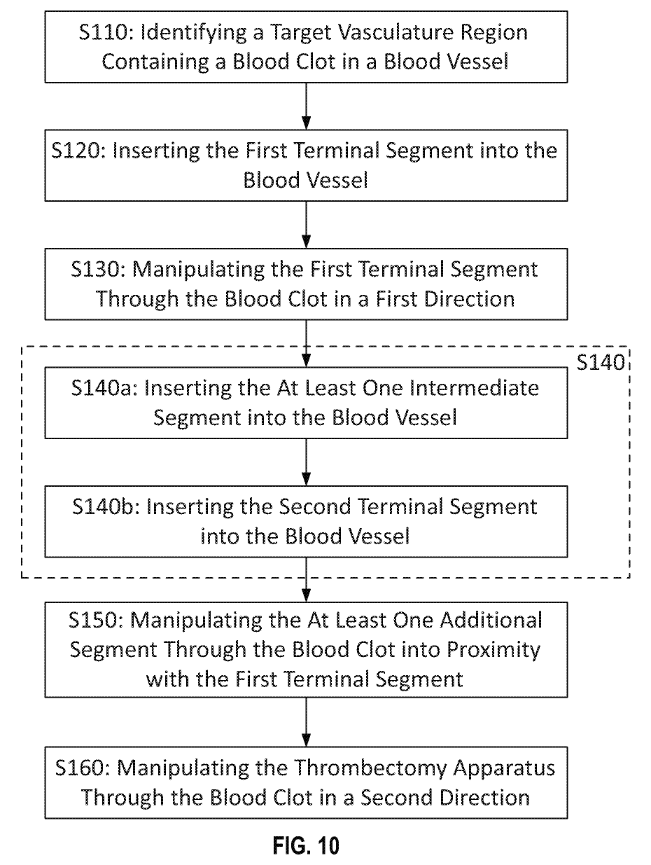

The following paragraphs include a discussion of items shown in FIG. 7 in conjunction with method steps (identified with parenthetical step numbers each beginning with the character “S” to denote a step) depicted in FIG. 9 and FIG. 10.

In use, a medical professional may identify (e.g., using a suitable imaging device) a target vasculature region (S110) of the animal body including a blood clot 102 (see FIG. 7), before inserting a first terminal segment (S120) into a blood vessel 100 of the target vasculature region. Such insertion may be performed using a catheter or similar device (not shown). The first terminal segment 6 is preferably inserted such that the tapered portion 14 is oriented toward a blood clot 102 in the blood vessel 100. Once the first terminal segment 6 is inserted, the first terminal segment 6 is magnetically manipulated (S130) to permit the tapered portion 14 thereof to pierce the blood clot 102 in a first direction and to permit the first terminal segment 6 to transit through the blood clot 102. The first terminal segment 6 is manipulated by interaction with a magnetic field generated by one or more magnetic field sources 34-1 to 34-3, which are located external to an animal body containing the blood vessel 100. The magnetic field source(s) 34-1 to 34-3 may be moved along the blood vessel 100 (e.g., using a corresponding robotic arm or similar device for each magnetic field source) such that the first terminal segment 6 is manipulated and guided through the blood vessel 100 toward and through the blood clot 102. Each magnetic field source 34-1 to 34-3 includes a corresponding pair of diametrically opposed magnetic field source generator (e.g., field source 34-1 includes opposing magnetic field source generators 34-1A, 34-1B; field source 34-2 includes opposing magnetic field source generators 34-2A, 34-2B; and field source 34-3 includes opposing magnetic field source generators 34-3A, 34-3B), to permit each magnetic field source 34-1 to 34-3 to apply balanced magnetic forces to one or more segments 6, 8, 32 (or a mated segment assembly 2) in a desired direction, without application of net force in a direction that would tend to pull a segment or mated segment assembly directly toward a single magnetic field source generator 34-1A, 34-1B, 34-2A, 34-2B, 34-3A, 34-3B.

After the first terminal segment 6 passes through the blood clot 102, at least one additional segment (e.g., intermediate segment 8 and/or second terminal segment 32) is inserted (S140) into the blood vessel 100. Each additional segment (e.g., intermediate segment 8) is manipulated (S150) and guided through the blood vessel 100 and blood clot 102 by the one or more magnetic field sources 34-1 to 34-3 into proximity with the first terminal segment 6. When brought into proximity with the first terminal segment 6, the at least one additional segment (e.g., intermediate segment 8) presses against and is magnetically coupled to the first terminal segment 6, such that the plurality of projections 18 of adjacent segments are outwardly deflected, from a relaxed first state to a deflected second state.

When the first terminal segment 6 and the at least one additional segment (e.g., intermediate segment 8 and/or second terminal segment 32) are magnetically coupled, the resulting mated segment assembly 2 is moved by the one or more magnetic field sources 34-1 to 34-3 in a second direction (opposite to the first direction) back to the blood clot 102 (S160). With the plurality of projections 18 between segments 6, (8 and/or 32) in the second state (being deflected outwardly relative to the respective first terminal segment longitudinal axis 12 and the additional segment longitudinal axis 26, to increase a maximum width or diameter of the segments 6, (8 and/or 32), the segments 6, (8 and/or 32) form a mated segment assembly 2 that engages the blood clot 102. Continued application of magnetic force to the mated segment assembly 2 using the one or more magnetic field sources 34-1 to 34-3 dislodges the blood clot 102 from the blood vessel 100 to permit the blood clot 102 to be removed (e.g., via catheter, such as the same catheter that deployed the segments 6, (8 and/or 32) into the blood vessel 100).

When the at least one additional segment includes at least one intermediate segment 8 and the second terminal segment 32, the at least one intermediate element 8 is inserted (S140a) into the blood vessel 100 prior to insertion of the second terminal segment 32 (S140b), such that the at least one intermediate segment 8 is located between the first terminal segment 6 and the second terminal segment 32. The second terminal segment 32 is inserted in an orientation such that the tapered portion 14 points away from the blood clot 102 at the time of insertion, so that when the mated segment assembly 2 is moved in the second direction (S160) the tapered portion 14 of the second terminal segment 32 is received within the opening predefined in the blood clot 102 to assist with centering of the mated segment assembly 2 relative to the blood clot 102 for engagement thereof.

FIG. 8 is a perspective view of an intermediate segment 8 with projections 18 (arranged along end portions 28 of an additional segment body structure 24) arranged an outwardly deflected state. As shown, the additional segment body structure 24 is in a curved shape, consistent with the flexible character of the body structure 24 to permit navigation of curved and/or tortuous paths within blood vessels of an animal body. The projections 18 are radially spaced along a perimeter of end portions 28 of the additional segment body structure 24. Although no other segment is shown in FIG. 8, it is to be understood that the projections 18 would typically be in a relaxed state (e.g., substantially parallel to a longitudinal axis of the additional segment body structure when relaxed) unless and until the intermediate segment 8 would contact other segments along ends thereof to cause outward deflection of the projections 18. Presence of spaces between adjacent projections 18 is useful to permit blood to continue to flow past a mated segment assembly even when the projections 18 are in a deflected state.

Those skilled in the art will recognize improvements and modifications to the preferred embodiments of the present disclosure. All such improvements and modifications are considered within the scope of the concepts disclosed herein and the claims that follow.

What is claimed is:

1. A thrombectomy apparatus configured to permit removal of a blood clot from a blood vessel, the apparatus comprising:
a plurality of segments configured to be moved within a blood vessel of an animal body responsive to application of magnetic fields by one or more magnetic field generators external to the animal body, wherein the plurality of segments comprises:
a first terminal segment comprising at least one first magnetically responsive element arranged within a first terminal segment body structure having a first terminal segment longitudinal axis, the first terminal segment body structure comprising a tapered end portion and an expandable terminal segment end portion that opposes the tapered end portion, the expandable terminal segment end portion comprising a plurality of projections extending substantially parallel to the first terminal segment longitudinal axis; and
at least one additional segment, wherein each additional segment of the at least one additional segment comprises at least one second magnetically responsive element arranged within an additional segment body structure having an additional segment longitudinal axis and at least one expandable additional segment end portion that comprises a plurality of projections extending substantially parallel to the additional segment longitudinal axis;
wherein upon magnetic manipulation of the first terminal segment and an additional segment of the at least one additional segment to press against one another, the plurality of projections of the expandable terminal segment end portion are configured to cooperate with the plurality of projections of the at least one expandable additional segment end portion to deflect outwardly relative to the first terminal segment longitudinal axis and the additional segment longitudinal axis, respectively.

2. The apparatus of claim 1, wherein the at least one magnetically responsive element of the first terminal segment and/or the at least one second magnetically responsive element of the at least one additional segment is spherical or cylindrical in shape.

3. The apparatus of claim 1, wherein:
the at least one additional segment comprises multiple additional segments, and
upon magnetic manipulation of each additional segment of the multiple additional segments to press against one another, the plurality of projections of each additional segment are configured to cooperate with the plurality of projections of an adjacent additional segment to deflect outwardly relative to the respective additional segment longitudinal axis.

4. The apparatus of claim 1, wherein:
the at least one additional segment comprises a second terminal segment for which the additional segment body structure comprises a second terminal segment body structure having the additional segment longitudinal axis, the second terminal segment body structure comprising a second tapered end portion that opposes the at least one expandable additional segment end portion thereof, and
upon magnetic manipulation of the first terminal segment and the second terminal segment to press against one another, the plurality of projections of the expandable terminal segment end portion of the first terminal segment are configured to cooperate with the plurality of projections of the at least one expandable additional segment end portion of the second terminal segment to deflect outwardly relative to the first terminal segment longitudinal axis and the additional segment longitudinal axis, respectively.

5. The apparatus of claim 1, wherein:
the at least one additional segment comprises at least two additional segments, the at least two additional segments including a second terminal segment and at least one intermediate segment that is arranged to be positioned between the first terminal segment and the second terminal segment;
the additional segment body structure of the second terminal segment comprises a second terminal segment body structure having the additional segment longitudinal axis, the second terminal segment body structure comprising a second tapered end portion and that opposes the at least one additional segment end portion thereof, and
upon magnetic manipulation of the first terminal segment, the at least one intermediate segment, and the second terminal segment to press against one another, the plurality of projections of the expandable terminal segment end portion of the first terminal segment are configured to cooperate with the plurality of projections of one expandable additional segment end portion of at the at least one intermediate segment to deflect outwardly relative to the first terminal segment longitudinal axis, and the plurality of projections of one expandable additional segment end portion of the second terminal segment are configured to cooperate with the plurality of projections of another expandable segment end portion of the at least one intermediate segment to deflect outwardly relative to the additional segment longitudinal axis.

6. The apparatus of claim 1, wherein the tapered end portion of the first terminal segment is formed from a material having a durometer value greater than durometer value of a material forming the first terminal segment body structure of the first terminal end segment.

7. The apparatus of claim 4, wherein the tapered end portion of the second terminal segment is formed from a material having a durometer value greater than a durometer value of a material forming the second terminal segment body structure of the second terminal end segment.

8. The apparatus of claim 5, wherein the tapered end portion of the second terminal segment is formed from a material having a durometer value greater than a durometer value of a material forming the second terminal segment body structure of the second terminal end segment.

9. A method of removing a blood clot from a blood vessel of an animal body utilizing a thrombectomy apparatus according to claim 1, the method comprising:
identifying a target vasculature region of the animal body including a blood vessel containing the blood clot;
inserting the first terminal segment into the target vasculature region;
moving, by the one or more magnetic field generators external to the animal body, the first terminal segment within the blood vessel and through the blood clot in a first direction;
inserting the at least one additional segment into the target vasculature region;
moving, by the one or more magnetic field generators, the at least one additional segment within the blood vessel and through the blood clot in the first direction to a position proximate to the first terminal segment and causing first terminal segment and the at least one additional segment to press against one another, thereby causing the pluralities of projections thereof to deflect outwardly relative to the first terminal segment longitudinal axis and the additional segment longitudinal axis to expand the expandable terminal end segment portion and to expand the at least one expandable additional segment end portion, respectively, and to yield a mated segment assembly; and
moving, by the one or more magnetic field generators, the mated segment assembly in a second direction that opposes the first direction, to cause the outwardly deflected pluralities of projections to engage the blood clot and dislodge at least a portion of the blood clot from the blood vessel.

10. The method of claim 9, wherein the inserting of the first terminal segment into the vasculature region further comprises:
prior to inserting the first terminal segment into the target vasculature region, orienting the first terminal segment such that the tapered end portion faces the blood clot in the first direction.

11. The method of claim 9, wherein:
the at least one additional segment comprises at least two additional segments, the at least two additional segments comprising a second terminal segment and at least one intermediate segment that is arranged to be positioned between the first terminal segment and the second terminal segment; the additional segment body structure of the second terminal segment comprises a second terminal segment body structure having the additional segment longitudinal axis, the second terminal segment body structure comprising a second tapered end portion that opposes the at least one expandable additional segment end portion thereof; and
the inserting of the at least one additional segment into the target vasculature region further comprises:
inserting the at least one intermediate segment into the target vascular region prior to inserting the second terminal segment into the target vascular region.

12. The method of claim 11, wherein the inserting of the second terminal segment further comprises:
prior to inserting the second terminal segment into the target vascular region, orienting the second terminal segment such that the tapered end portion faces away from the blood clot.

13. The method of claim 9, wherein each field generator of the one or more external field generators comprises a pair of magnetic field sources, the pair of magnetic field sources being diametrically opposed to one another around the target vasculature region.
